(19) United States
(12) Patent Application Publication
(10) Pub. No.: US 2022/0411451 A1
(43) Pub. Date: Dec. 29, 2022

(54) ACTIVE HETEROGENIZED PALLADIUM-BRIDGED-NHC CATALYSTS FOR CARBONYLATIVE SONOGASHIRA COUPLING REACTIONS

(71) Applicants: Saudi Arabian Oil Company, Dhahran (SA); King Fahd University of Petroleum & Minerals, Dhahran (SA)

(72) Inventors: Bassam El Ali, Dhahran (SA); Waseem Mansour, Dhahran (SA); Mohammed Fettouhi, Dhahran (SA)

(21) Appl. No.: 17/329,647

(22) Filed: May 25, 2021

(51) Int. Cl. C07F 15/00; B01J 31/22; C07D 311/54

(52) U.S. Cl. CPC C07F 15/0066; B01J 31/2273; C07D 311/54; B01J 2531/824; B01J 2231/321

(57) ABSTRACT

This disclosure relates to solid-supported bridged Pd(II)—N-heterocyclic carbene catalysts, methods of preparing the catalysts, and methods of using the catalysts in carbonylative Sonogashira coupling reactions.

### TECHNICAL FIELD

This document relates to solid-supported bridged Pd(II)—N-heterocyclic carbene (NHC) catalysts. The document also relates to use of the catalysts in carbonylative Sonogashira coupling reactions to form alkynones and cyclocarbonylative Sonogashira coupling reactions to form chromones.

### BACKGROUND

Alkynyl ketones, or alkynones, and chromones have utility as synthetic intermediates, particularly for the synthesis of heterocyclic systems that can be used as precursors in the synthesis of anti-fungal products, as well as products useful for treating neurodegenerative, inflammatory, and infectious diseases, diabetes, and cancer. Alkynones and chromones have also found use in the polymer industry and petrochemical industry. A common route for the synthesis of these compounds involves a carbonylative or cyclocarbonylative Sonogashira coupling reaction. These reactions are typically catalyzed by a palladium complex and often require a large excess of an amine base. High catalyst loading, for example, greater than 2 mol % of the palladium complex is often required. This can lead to higher costs and less efficient reactions.

Therefore, there is a need for a palladium catalyst that can catalyze a Sonogashira coupling reaction, in particular, a carbonylative or cyclocarbonylative Sonogashira coupling reaction, that has high catalytic activity, is stable, requires low catalyst loading, and is recyclable.

### SUMMARY

Provided in the present disclosure is a compound of Formula (I):

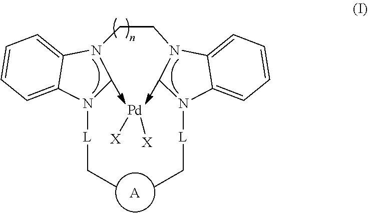

wherein: L is absent or is selected from the group consisting of —C₁-C₆ alkyl- and —C₁-C₆ alkyl—O—; X is selected from the group consisting of Cl, Br, and I; A is a solid support; and n is 0 to 4.

In some embodiments of the compound of Formula (I), L is —C₁-C₆ alkyl—O—. In some embodiments, L is selected from —CH₂—O—, —(CH₂)₂—O—, —(CH₂)₃—O—, —(CH₂)₄—O—, —(CH₂)₅—O—, and —(CH₂)₆—O—. In some embodiments, L is —(CH₂)₃—O—. In some embodiments, L is absent.

In some embodiments of the compound of Formula (I), X is Cl.

In some embodiments of the compound of Formula (I), A is Merrifield resin.

In some embodiments of the compound of Formula (I), n is 1 or 2. In some embodiments, n is 2.

In some embodiments, the compound of Formula (I) is selected from:

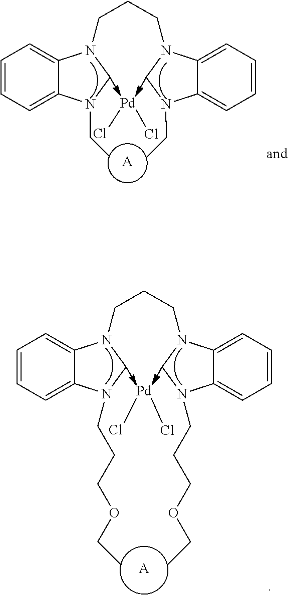

In some embodiments, the compound of Formula (I) has a turnover number in a range of about 1500 to about 2500 and a turnover frequency of about 200 to about 1500 per hour.

Also provided is a compound of Formula (II)

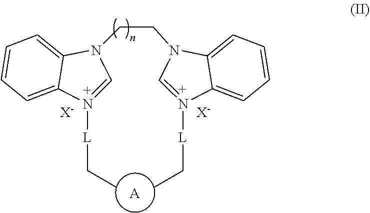

wherein: L is absent or is selected from the group consisting of —C₁-C₆ alkyl- and —C₁-C₆ alkyl—O—; X is selected from the group consisting of Cl, Br, and I; A is a solid support; and n is 0 to 4.

In some embodiments of the compound of Formula (II), L is —C₁-C₆ alkyl—O—. In some embodiments, L is selected from —CH₂—O—, —(CH₂)₂—O—, —(CH₂)₃—O—, —(CH₂)₄—O—, —(CH₂)₅—O—, and —(CH₂)₆—O—. In some embodiments, L is —(CH₂)₃—O—. In some embodiments, L is absent.

In some embodiments of the compound of Formula (II), A is Merrifield resin.

In some embodiments of the compound of Formula (II), n is 2.

In some embodiments the compound of Formula (II) is selected from:

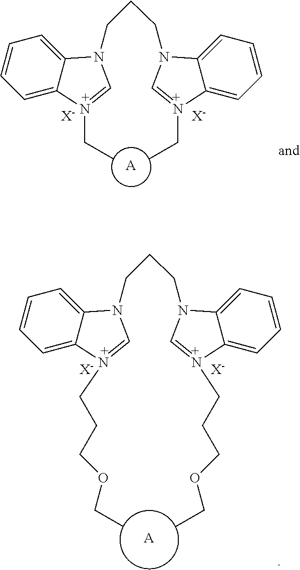

Also provided in the present disclosure is a method of preparing a compound of Formula (I)

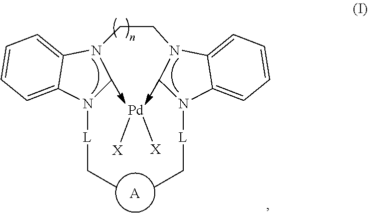

the method comprising reacting a compound of Formula (II)

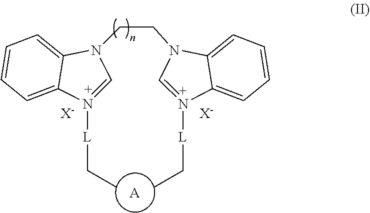

with a palladium catalyst, wherein: L is absent or is selected from the group consisting of—C₁-C₆ alkyl- and —C₁-C₆ alkyl—O—; X is selected from the group consisting of Cl, Br, and I; A is a solid support; and n is 0 to 4.

In some embodiments of the method, the palladium catalyst is palladium acetate.

In some embodiments, the compound of Formula (I) is selected from:

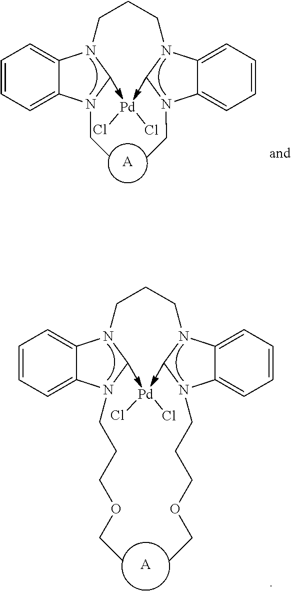

Also provided is a method of preparing an alkynone, or a pharmaceutically acceptable salt thereof, comprising contacting an aryl halide and an alkyne with a compound of Formula (I) according to claim 1 in the presence of a CO source to form the alkynone, or a pharmaceutically acceptable salt thereof.

In some embodiments of the method, the aryl halide is a compound having the formula:

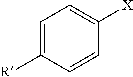

wherein: X is selected from F, Cl, Br, and I; and R′ is selected from H, C₁-C₆ alkyl, —O—(C₁—C₆ alkyl), —C(═O)C₁-C₆ alkyl, —CN, and aryl.

In some embodiments of the method, X is I.

In some embodiments of the method, the alkyne is a compound having the formula:

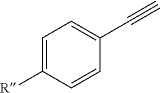

wherein R″ is selected from H, C₁-C₆ alkyl, —O—(C₁-C₆ alkyl), —C(═O)C₁-C₆ alkyl, and aryl.

In some embodiments of the method, the alkyne is a compound having the formula:

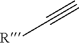

wherein R′″ is selected from C₁-C₆ alkyl and C₃-C₉ cycloalkyl.

In some embodiments of the method, the alkynone is a compound having the formula:

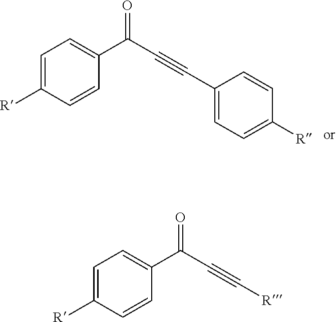

or a pharmaceutically acceptable salt thereof.

In some embodiments of the method, the compound of Formula (I) is selected from:

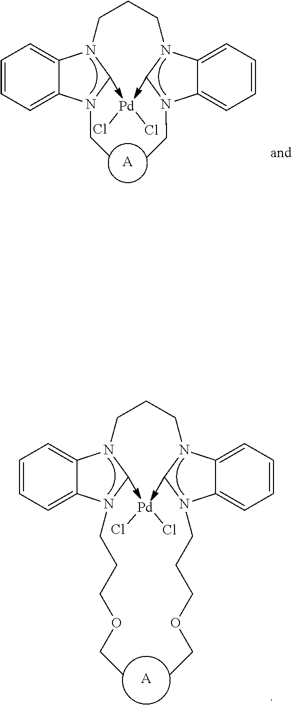

In some embodiments of the method, the compound of Formula (I) is present in an amount of about 0.01 mol % to about 1.0 mol %.

In some embodiments the method further comprises: separating the compound of Formula (I) from the alkynone, or pharmaceutically acceptable salt thereof, to recover the compound of Formula (I); and reusing the compound of Formula (I) in at least 2 reaction cycles with a less than about 10% decrease in at least one selected from the group consisting of a turnover number and a turnover frequency.

Also provided in the present disclosure is a method of preparing a chromone, or a pharmaceutically acceptable salt thereof, comprising contacting a 2-iodophenol and an alkyne with a compound of Formula (I) according to claim 1 in the presence of a CO source to form the chromone, or a pharmaceutically acceptable salt thereof.

In some embodiments of the method, the 2-iodophenol is a compound having the formula:

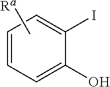

wherein R^{a} is selected from H, C₁-C₆ alkyl, —O—(C₁-C₆ alkyl), —C(═O)C₁-C₆ alkyl, and aryl.

In some embodiments of the method, R^{a} is H.

In some embodiments of the method, the alkyne is a compound having the formula:

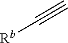

wherein R^{b} is selected from C₁-C₆ alkyl, aryl.

In some embodiments of the method, the alkyne is a compound having the formula:

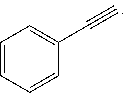

In some embodiments of the method, the chromone is a compound having the formula:

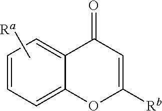

or a pharmaceutically acceptable salt thereof.

In some embodiments of the method, the compound of Formula (I) is selected from:

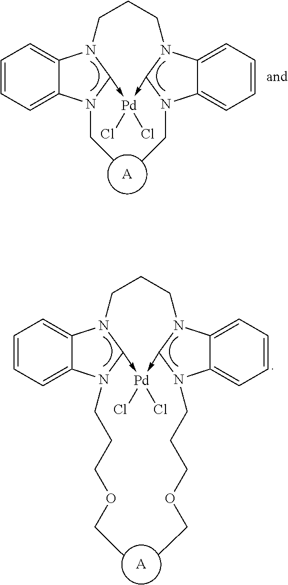

In some embodiments of the method, the compound of Formula (I) is present in an amount of about 0.01 mol % to about 2.0 mol %.

In some embodiments the method further comprises: separating the compound of Formula (I) from the chromone, or pharmaceutically acceptable salt thereof, to recover the compound of Formula (I); and reusing the compound of Formula (I) in at least 2 reaction cycles with a less than about 10% decrease in at least one selected from the group consisting of a turnover number and a turnover frequency.

### DETAILED DESCRIPTION

The present disclosure relates to solid-supported bridged N-heterocyclic carbene (NHC) ligands and solid-supported bridged Pd(II) NHC catalysts. The solid-supported bridged Pd(II) NHC catalysts exhibit high catalytic activity and efficiency with low catalyst loading. For example, the solid-supported bridged Pd(II) NHC catalysts exhibit high catalytic efficiency in the synthesis of alkynones via carbonylative Sonogashira coupling reactions and chromones via cyclocarbonylative Sonogashira coupling reactions. In some embodiments, the carbonylative Sonogashira coupling reaction is between an aryl halide or aryl dihalide with an aryl alkyne, alkyl alkyne, or dialkyne. In some embodiments, the cyclocarbonylative Sonogashira coupling reaction is between an aryl iodide with an aryl alkyne. In some embodiments, the reaction is a one pot reaction. The resulting alkynones and chromones can be useful precursors in the synthesis of anti-fungal products, as well as products useful for treating neurodegenerative, inflammatory, and infectious diseases, diabetes, and cancer. The alkynones and chromones can also be used in the polymer and petrochemical industries.

The catalysts of the present disclosure are also recyclable. For example, the catalysts of the present disclosure can be recovered and reused (recycled) in subsequent chemical reactions. In some embodiments, the catalyst is able to be reused and recycled without significantly losing any catalytic activity in a variety of chemical reactions. The recycling of homogeneous catalysts is complex and costly. Therefore, the use of an immobilized catalyst is an alternative for industries to combine the advantages of both homogeneous and heterogeneous catalysts and also to overcome the problems related to metal contamination.

Thus, one object of the present disclosure is to provide a solid-supported bridged NHC ligand having suitable functionality for coordinating palladium (II) and a heterogeneous solid-supported bridged NHC palladium (II) catalyst thereof. In some embodiments, the catalyst is a Merrifield resin-supported palladium bis(NHC) catalyst. A further object of the present disclosure is to provide methods for preparing the solid-supported ligand and solid-supported palladium (II) catalyst as well as methods employing the solid-supported catalyst in palladium cross-coupling reactions, such as carbonylative and cyclocarbonylative Sonogashira reactions, demonstrating significant stability, catalytic activity, and recycling ability.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

In this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used herein can allow for a degree of variability in a value or range, for example, within 10%, within 5%, or within 1% of a stated value or of a stated limit of a range.

In the methods described in the present disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

Compounds of Formula (I)

Provided in the present disclosure is a compound of Formula (I)

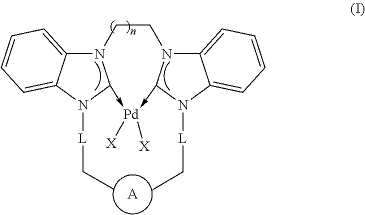

wherein:

L is absent or is selected from the group consisting of —C₁-C₆ alkyl- and —C₁-C₆ alkyl-O—;

X is selected from the group consisting of Cl, Br, and I;

A is a solid support; and

n is 0 to 4.

In some embodiments of the compound of Formula (I), L is absent.

In some embodiments of the compound of Formula (I), L is selected from the group consisting of —C₁-C₆ alkyl- and —C₁-C₆ alkyl—O—. In some embodiments, L is —C₁-C₆ alkyl-. In some embodiments, L is methyl. In some embodiments, L is ethyl. In some embodiments, L is propyl. In some embodiments, L is isopropyl. In some embodiments, L is butyl. In some embodiments, L is —C₁-C₆ alkyl—O—. In some embodiments, L is selected from —CH₂—O—, —(CH₂)₂—O—, —(CH₂)₃—O—, —(CH₂)₄—O—, —(CH₂)₅—O—, and —(CH₂)₆—O—. In some embodiments, L is —CH₂—O—. In some embodiments, L is —(CH₂)₂—O—. In some embodiments, L is —(CH₂)₃—O—. In some embodiments, L is —(CH₂)₄—O—. In some embodiments, L is —(CH₂)₅—O—. In some embodiments, L is —(CH₂)₆—O—.

In some embodiments of the compound of Formula (I), X is Cl. In some embodiments of the compound of Formula (I), X is Br. In some embodiments of the compound of Formula (I), X is I.

In some embodiments of the compound of Formula (I), A represents a solid support. As used herein, a solid support is a material, usually a solid with a high surface area, to which a catalyst or ligand is affixed. The activity of heterogeneous catalysts and nanomaterial-based catalysts occurs at the surface atoms. The solid support can be inert or can participate in catalytic reactions. In some embodiments, the solid support is inert.

In some embodiments, the solid support is functionalized to facilitate covalent attachment of the bridged NHC ligand of the present disclosure. As used herein, the term “functionalize” refers to modification of a surface of the solid support particle with an organic moiety containing carbon. Exemplary organic moieties include, but are not limited to, 4-benzyl chloride, 3-aminopropyl, 4-bromopropyl, 4-bromophenyl, 3-carboxypropyl, 2-(carbomethoxy)propyl, 3-chloropropyl, 3-(2-succinic anhydride)propyl, 1-(allyl)methyl, 3-(thiocyano)propyl, 3-(isocyano)propyl, propionyl chloride, 3-(maleimido)propyl, 3-(glycidoxy)propyl, 4-ethyl benzenesulfonyl chloride, 2-(3,4-epoxycyclohexyl)propyl, and 3-propylsulfonic acid. In some embodiments, the solid support is functionalized with 4-benzyl chloride. Loading of the organic moiety on the solid support can be from about 0.5 mmol/g to about 20 mmol/g, about 1 mmol/g to about 10 mmol/g, about 1 mmol/g to about 5 mmol/g, or about 1 mmol/g to about 3 mmol/g.

The solid support can be any solid support known to one of skill in the art that can immobilize a catalyst or ligand, such as a catalyst or ligand of the present disclosure. In some embodiments, the catalyst is immobilized by covalent coupling to a grafted or a functionalized polystyrene support. Exemplary functionalized polystyrene supports include, but are not limited to, Merrifield resin, Wang resin, Argogel resin, Tentagel resin, and polyamine resins. In some embodiments, the functionalized polystyrene support is Merrifield resin. In some embodiments, the polystyrene support is Merrifield resin functionalized with 4-benzyl chloride. In some embodiments, the catalyst is immobilized by covalent coupling to a grafted or a functionalized polymer support, wherein the functionalized polymer support is at least one selected from the group consisting of polyolefins, polyacrylates, polymethacrylates, and copolymers thereof.

In some embodiments, the solid support is Merrifield resin. As used herein, Merrifield resin refers to a cross-linked polystyrene resin that carries a chloromethyl functional group. Merrifield resin is a polystyrene resin based on a copolymer of styrene and chloromethyl styrene. This polymer can further be cross-linked with divinyl benzene, wherein a degree of crosslinking is within the range of about 1% to about 5%, such as about 1% to about 2%. In some embodiments, the solid support comprises at least about 10 wt % Merrifield resin relative to the total weight of the solid support, such as at least about 50 wt %, at least about 70 wt %, at least about 80 wt %, at least about 90 wt %, at least about 95 wt %, at least about 96 wt %, at least about 97 wt %, at least about 98 wt %, or at least about 99 wt % Merrifield resin relative to the total weight of the solid support. In some embodiments, one or more solid supports are used in addition to, or instead of, Merrifield resin.

In some embodiments, the catalyst is immobilized by covalent coupling, such as through a silicon- or siloxane-containing linker, to a porous or nonporous solid support. Exemplary possible supports include, but are not limited to, alumina, titanium, kieselguhr, diatomaceous earth, clay, zeolites, carbon black, activated carbon, graphite, fluorinated carbon, organic polymers, metals, metal alloys, and mixtures thereof.

In some embodiments of the compound of Formula (I), A is Merrifield resin.

In some embodiments of the compound of Formula (I), n is 0. In some embodiments, n is 1 or 2. In some embodiments, n is 1. In some embodiments, n is 2. In some embodiments, n is 3. In some embodiments, n is 4.

In some embodiments, the compound of Formula (I) is a compound of Formula (Ia):

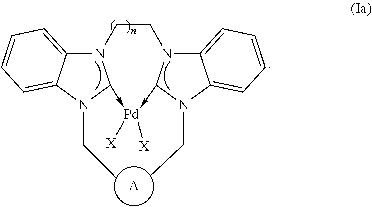

In some embodiments, the compound of Formula (I) is a compound of Formula (Ib):

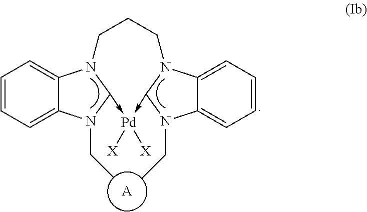

In some embodiments, the compound of Formula (I) is a compound of Formula (Ic):

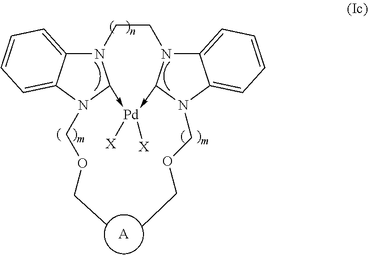

wherein m is 1 to 6. In some embodiments, m is 1. In some embodiments, m is 2. In some embodiments, m is 3. In some embodiments, m is 4. In some embodiments, m is 5. In some embodiments, m is 6.

In some embodiments, the compound of Formula (I) is a compound of Formula (Id):

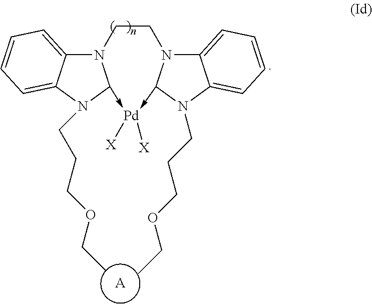

In some embodiments, the compound of Formula (I) is a compound of Formula (Ie):

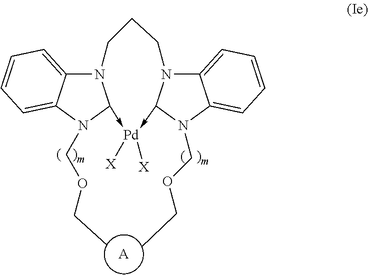

wherein m is 1 to 6. In some embodiments, m is 1. In some embodiments, m is 2. In some embodiments, m is 3. In some embodiments, m is 4. In some embodiments, m is 5. In some embodiments, m is 6.

In some embodiments, the compound of Formula (I) is a compound of Formula (If):

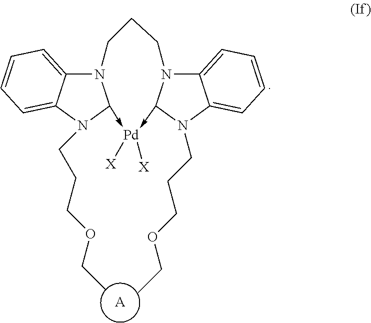

In some embodiments, the compound of Formula (I) is selected from:

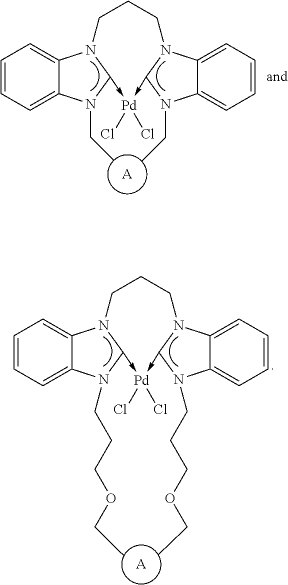

In some embodiments, the compound of Formula (I) is

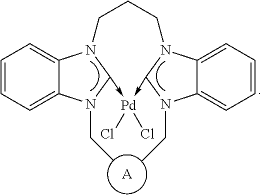

In some embodiments, the compound of Formula (I) is

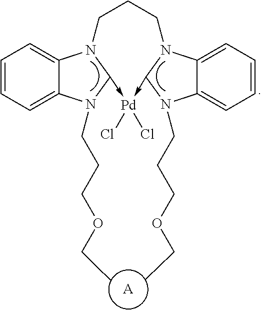

Compounds of Formula (II)

Also provided in the present disclosure are compounds of Formula (II)

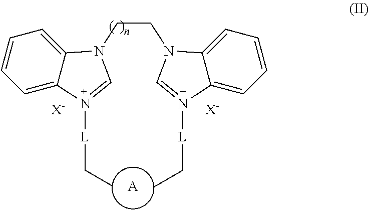

wherein:

L is absent or is selected from the group consisting of —C₁-C₆ alkyl- and —C₁-C₆ alkyl-0-;

X is selected from the group consisting of Cl, Br, and I;

A is a solid support; and

n is 0 to 4.

In some embodiments of the compound of Formula (II), L is absent.

In some embodiments of the compound of Formula (II), L is selected from the group consisting of —C₁-C₆ alkyl- and —C₁-C₆ alkyl—O—. In some embodiments, L is —C₁-C₆ alkyl-. In some embodiments, L is methyl. In some embodiments, L is ethyl. In some embodiments, L is propyl. In some embodiments, L is isopropyl. In some embodiments, L is butyl. In some embodiments, L is —C₁-C₆ alkyl—O—. In some embodiments, L is selected from —CH₂—O—, —(CH₂)₂—O—, —(CH₂)₃—O—, —(CH₂)₄—O—, —(CH₂)₅—O—, and —(CH₂)₆—O—. In some embodiments, L is —CH₂—O—. In some embodiments, L is —(CH₂)₂—O—. In some embodiments, L is —(CH₂)₃—O—. In some embodiments, L is —(CH₂)₄—O—. In some embodiments, L is —(CH₂)₅—O—. In some embodiments, L is —(CH₂)₆—O—.

In some embodiments of the compound of Formula (II), X is Cl. In some embodiments of the compound of Formula (II), X is Br. In some embodiments of the compound of Formula (II), X is I.

In some embodiments of the compound of Formula (II), A represents a solid support. In some embodiment, A is Merrifield resin.

In some embodiments of the compound of Formula (II), n is 0. In some embodiments,
n is 1 or 2. In some embodiments, n is 1. In some embodiments, n is 2. In some embodiments, n is 3. In some embodiments, n is 4.

In some embodiments, the compound of Formula (II) is a compound of Formula (IIa):

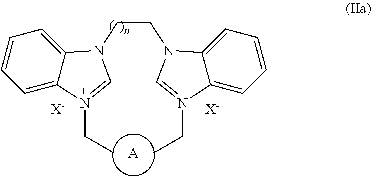

In some embodiments, the compound of Formula (II) is a compound of Formula (IIb):

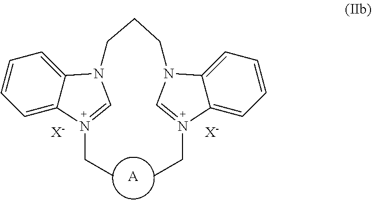

In some embodiments, the compound of Formula (II) is a compound of Formula (IIc):

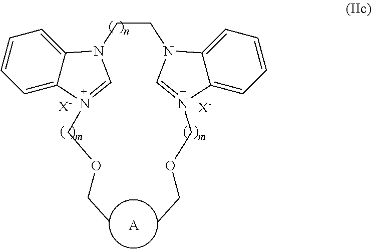

wherein m is 1 to 6. In some embodiments, m is 1. In some embodiments, m is 2. In some embodiments, m is 3. In some embodiments, m is 4. In some embodiments, m is 5. In some embodiments, m is 6.

In some embodiments, the compound of Formula (II) is a compound of Formula (IId):

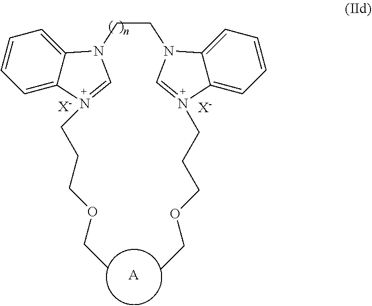

In some embodiments, the compound of Formula (II) is a compound of Formula (IIe):

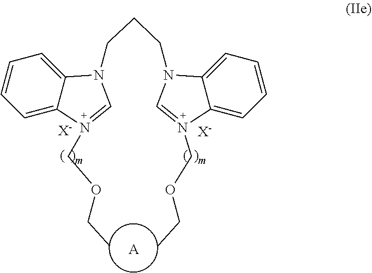

wherein m is 1 to 6. In some embodiments, m is 1. In some embodiments, m is 2. In some embodiments, m is 3. In some embodiments, m is 4. In some embodiments, m is 5. In some embodiments, m is 6.

In some embodiments, the compound of Formula (II) is a compound of Formula (IIf):

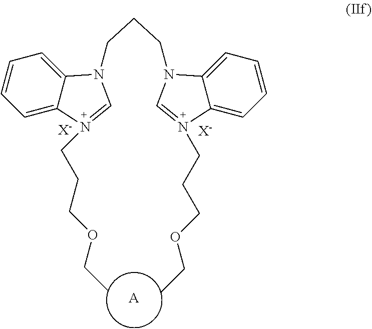

In some embodiments, the compound of Formula (II) is selected from:

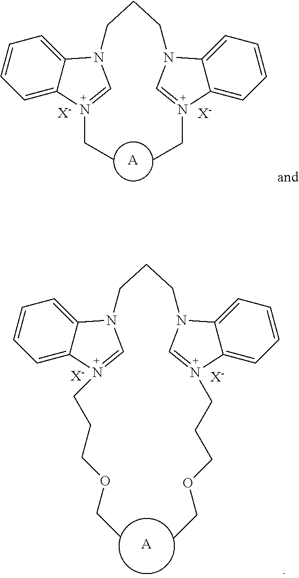

In some embodiments, the compound of Formula (II) is selected from:

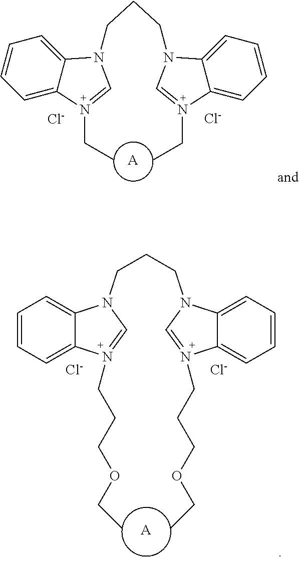

In some embodiments, the compound of Formula (II) is:

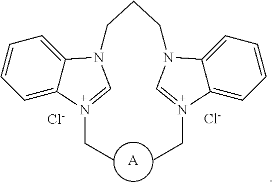

In some embodiments, the compound of Formula (II) is:

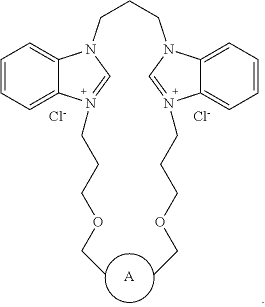

Method of preparing compounds of Formula (I) and Formula (II)

Also provided in the present disclosure are methods of preparing compounds of Formula (I) and Formula (II). In some embodiments, the method includes reacting a compound of Formula (II), such as a compound of Formula (II) as described in the present disclosure, with a palladium catalyst to form a compound of Formula (I). In some embodiments, the compound of Formula (I) is isolated. In some embodiments, the compound of Formula (I) is purified.

In some embodiments, the palladium catalyst is selected from the group consisting of palladium acetate, palladium bromide, palladium chloride, and palladium iodide. In some embodiments, the palladium catalyst is palladium acetate.

In some embodiments, the compounds of Formula (I) are prepared according to the general scheme presented in Scheme 1, where A, L, X, and n are as described elsewhere in this disclosure.

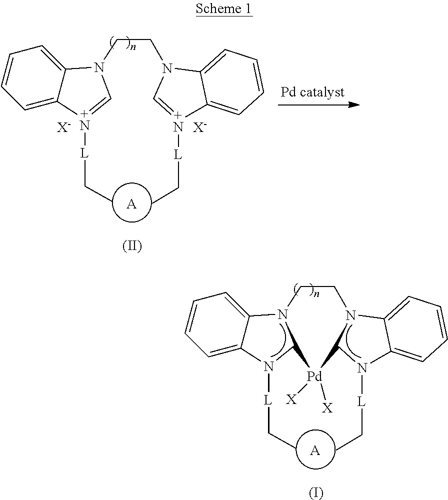

In some embodiments, the method includes preparing a compound of Formula (II). In some embodiments, the method includes reacting an alkylene-bridged bis(1H-benzo[d]imidazole) with a solid support resin to form a compound of Formula (II). In some embodiments, the alkylene-bridged bis(1H-benzo[d]imidazole) is functionalized. In some embodiments, the alkylene-bridged bis(1H-benzo[d]imidazole) is hydroxyl functionalized. In some embodiments, the solid support resin is a functionalized Merrifield resin support. In some embodiments, the solid support resin is chloro functionalized Merrifield resin support. In some embodiments, the compound of Formula (II) is isolated. In some embodiments, the compound of Formula (II) is purified. In some embodiments, the compound of Formula (II) is isolated and purified prior to using in the method of preparing compounds of Formula (I).

In some embodiments, the compound of Formula (II) is prepared according to the general scheme presented in Scheme 2a, where the method includes reacting an alkylene-bridged bis(1H-benzo[d]imidazole) with a functionalized solid support resin to form a compound of Formula (II), where A, X, and n are as described elsewhere in this disclosure.

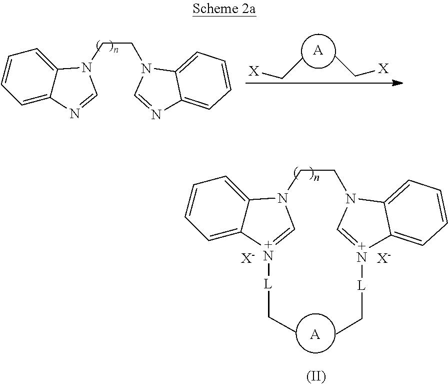

In some embodiments, the compound of Formula (II) is prepared according to the general scheme presented in Scheme 2b, where the method includes reacting a hydroxyl-functionalized alkylene-bridged bis(1H-benzo[d]imidazole) with a solid support resin to form a compound of Formula (II), where A, L, X, m, and n are as described elsewhere in this disclosure. In some embodiments, the method includes functionalizing an unsubstituted alkylene-bridged bis(1H-benzo[d]imidazole) prior to reacting the functionalized alkylene-bridged bis(1H-benzo[d]imidazole) with a solid support resin to form a compound of Formula (II).

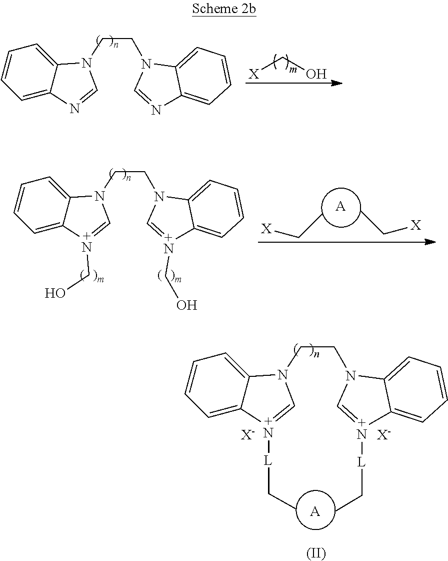

In some embodiments, the methods of the present disclosure are used to prepare a compound of Formula (I), where the compound of Formula (I) is selected from

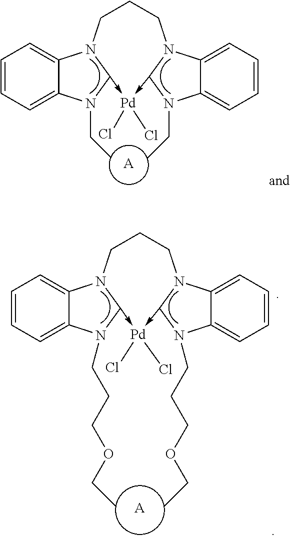

In some embodiments, the methods of the present disclosure are used to prepare a compound of Formula (II), where the compound of Formula (II) is selected from

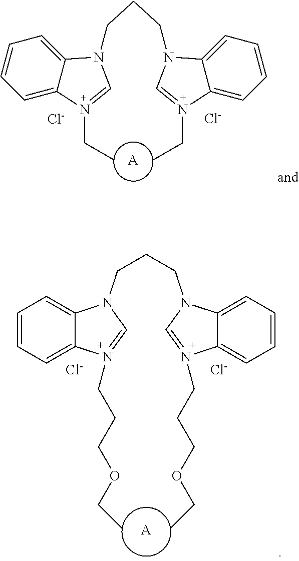

Methods of Preparing Alkynones

The compounds of Formula (I) of the present disclosure are useful as catalysts. For example, the compounds of Formula (I) can be used as catalysts for the synthesis of alkynones, including aryl alkynones and alkyl alkynones. In some embodiments, the compounds of Formula (I) are used as a catalyst in a carbonylative Sonogashira coupling reaction. In some embodiments, the carbonylative Sonogashira coupling reaction is between an aryl halide or aryl dihalide and an aryl alkyne, alkyl alkyne, or dialkyne. In some embodiments, the carbonylative Sonogashira coupling reaction is between an aryl bromide, aryl iodide, or aryl diiodide and an aryl alkyne, alkyl alkyne, or dialkyne.

In some embodiments, the alkynones of the present disclosure are prepared according to the general scheme presented in Scheme 3, where R—X can be an aryl halide, aryl dihalide, or vinyl halide, and R′—CC can be an aryl alkyne, alkyl alkyne, or dialkyne.

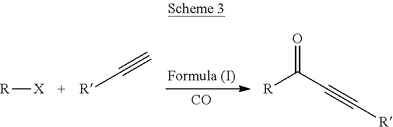

Thus, provided in the present disclosure is a method of preparing an alkynone, the method including contacting an aryl halide, aryl dihalide, or vinyl halide and an alkyne with a compound of Formula (I) as described in the present disclosure in the presence of a CO source.

In some embodiments, the method includes contacting an aryl halide and an alkyne with a compound of Formula (I) as described in the present disclosure in the presence of a CO source. In some embodiments, the aryl halide is a compound having the formula:

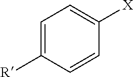

wherein:

X is selected from F, Cl, Br, and I; and

R′ is selected from H, C₁-C₆ alkyl, —O—(C₁-C₆ alkyl), —C(═O)C₁-C₆ alkyl, —CN, and aryl.

In some embodiments, Xis I.

In some embodiments, R′ is selected from H, —OCH₃, —C(═O)CH₃, and —CN.

In some embodiments, R′ is H. In some embodiments, R′ is —OCH₃. In some embodiments, R′ is —C(═O)CH₃. In some embodiments, R′ is —CN.

In some embodiments, the method includes contacting an aryl halide and an alkyne with a compound of Formula (I) as described in the present disclosure in the presence of a CO source. In some embodiments, the alkyne is a compound having the formula:

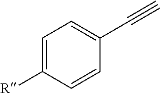

wherein R″ is selected from H, C₁-C₆ alkyl, —O—(C₁-C₆ alkyl), —C(═O)C₁-C₆ alkyl, and aryl.

In some embodiments, R″ is H. In some embodiments, R″ is —OCH₃. In some embodiments, R″ is C₁-C₆ alkyl. In some embodiments, R″ is methyl. In some embodiments, R″ is ethyl. In some embodiments, R″ is propyl. In some embodiments, R″ is butyl. In some embodiments, R″ is pentyl. In some embodiments, R″ is hexyl.

In some embodiments, the alkyne is a compound having the formula:

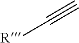

wherein R′ is selected from C₁-C₆ alkyl and C₃-C₉ cycloalkyl.

In some embodiments, R′ is C₁-C₆ alkyl. In some embodiments, R″ is methyl. In some embodiments, R″ is ethyl. In some embodiments, R″ is propyl. In some embodiments, R″ is butyl. In some embodiments, R″ is pentyl. In some embodiments, R″ is hexyl.

In some embodiments of the method of producing an alkynone, the compound of Formula (I) is a compound of Formula (I) of the present disclosure. In some embodiments, the compound of Formula (I) is selected from:

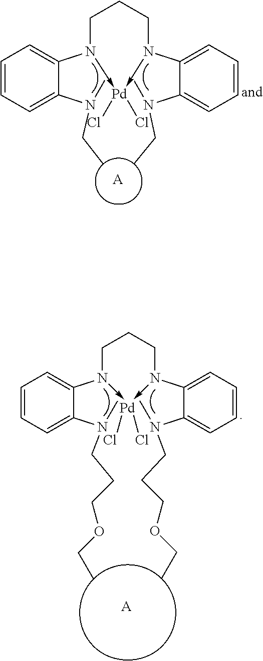

The compounds of Formula (I) have high catalytic efficiency and activity and allow for low catalyst loading. In some embodiments, less than or about 2 mol %, or less than or about 1 mol % of the compound of Formula (I) is required to catalyze a reaction, such as a carbonylative Sonogashira coupling reaction. In some embodiments, the amount of catalyst (compound of Formula (I)) used in the carbonylative Sonogashira coupling reaction is about 0.01 mol % to about 2 mol %, such as about 0.01 mol % to about 1 mol %, about 0.01 mol % to about 0.99 mol %, about 0.01 mol % to about 0.9 mol %, about 0.01 mol % to about 0.8 mol %, about 0.01 mol % to about 0.7 mol %, about 0.01 mol % to about 0.6 mol %, about 0.01 mol % to about 0.5 mol %, about 0.01 mol % to about 0.4 mol %, about 0.01 mol % to about 0.3 mol %, about 0.01 mol % to about 0.2 mol %, about 0.01 mol % to about 0.1 mol %, about 0.01 mol % to about 0.05 mol %, about 0.01 mol % to about 0.03 mol %, about 0.03 mol % to about 1 mol %, 0.03 mol % to about 0.99 mol %, about 0.03 mol % to about 0.9 mol %, about 0.03 mol % to about 0.8 mol %, about 0.03 mol % to about 0.7 mol %, about 0.03 mol % to about 0.6 mol %, about 0.03 mol % to about 0.5 mol %, about 0.03 mol % to about 0.4 mol %, about 0.03 mol % to about 0.3 mol %, about 0.03 mol % to about 0.2 mol %, about 0.03 mol % to about 0.1 mol %, about 0.03 mol % to about 0.05 mol %, about 0.05 mol % to about 1 mol %, 0.05 mol % to about 0.99 mol %, about 0.05 mol % to about 0.9 mol %, about 0.05 mol % to about 0.8 mol %, about 0.05 mol % to about 0.7 mol %, about 0.05 mol % to about 0.6 mol %, about 0.05 mol % to about 0.5 mol %, about 0.05 mol % to about 0.4 mol %, about 0.05 mol % to about 0.3 mol %, about 0.05 mol % to about 0.2 mol %, about 0.05 mol % to about 0.1 mol %, about 0.1 mol % to about 1 mol %, 0.1 mol % to about 0.99 mol %, about 0.1 mol % to about 0.9 mol %, about 0.1 mol % to about 0.8 mol %, about 0.1 mol % to about 0.7 mol %, about 0.1 mol % to about 0.6 mol %, about 0.1 mol % to about 0.5 mol %, about 0.1 mol % to about 0.4 mol %, about 0.1 mol % to about 0.3 mol %, about 0.1 mol % to about 0.2 mol %, about 0.2 mol % to about 1 mol %, 0.2 mol % to about 0.99 mol %, about 0.2 mol % to about 0.9 mol %, about 0.2 mol % to about 0.8 mol %, about 0.2 mol % to about 0.7 mol %, about 0.2 mol % to about 0.6 mol %, about 0.2 mol % to about 0.5 mol %, about 0.2 mol % to about 0.4 mol %, about 0.2 mol % to about 0.3 mol %, about 0.3 mol % to about 1 mol %, 0.3 mol % to about 0.99 mol %, about 0.3 mol % to about 0.9 mol %, about 0.3 mol % to about 0.8 mol %, about 0.3 mol % to about 0.7 mol %, about 0.3 mol % to about 0.6 mol %, about 0.3 mol % to about 0.5 mol %, about 0.3 mol % to about 0.4 mol %, about 0.4 mol % to about 1 mol %, 0.4 mol % to about 0.99 mol %, about 0.4 mol % to about 0.9 mol %, about 0.4 mol % to about 0.8 mol %, about 0.4 mol % to about 0.7 mol %, about 0.4 mol % to about 0.6 mol %, about 0.4 mol % to about 0.5 mol %, about 0.5 mol % to about 1 mol %, 0.5 mol % to about 0.99 mol %, about 0.5 mol % to about 0.9 mol %, about 0.5 mol % to about 0.8 mol %, about 0.5 mol % to about 0.7 mol %, about 0.5 mol % to about 0.6 mol %, about 0.6 mol % to about 1 mol %, 0.6 mol % to about 0.99 mol %, about 0.6 mol % to about 0.9 mol %, about 0.6 mol % to about 0.8 mol %, about 0.6 mol % to about 0.7 mol %, about 0.7 mol % to about 1 mol %, 0.7 mol % to about 0.99 mol %, about 0.7 mol % to about 0.9 mol %, about 0.7 mol % to about 0.8 mol %, about 0.8 mol % to about 1 mol %, 0.8 mol % to about 0.99 mol %, about 0.8 mol % to about 0.9 mol %, about 0.9 mol % to about 1 mol %, 0.9 mol % to about 0.99 mol %, or about 0.01 mol %, about 0.03 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.55 mol %, about 0.6 mol %, about 0.65 mol %, about 0.7 mol %, about 0.75 mol %, about 0.8 mol %, about 0.85 mol %, about 0.9 mol %, about 0.95 mol %, about 0.99 mol %, or about 1 mol %. In some embodiments, the compound of Formula (I) is present in an amount of about 0.01 mol % to about 1.0 mol %. In some embodiments, the compound of Formula (I) is present in an amount of about 0.01 mol % to about 0.5 mol %. In some embodiments, the compound of Formula (I) is present in an amount of about 0.01 mol % to about 0.05 mol %. In some embodiments, the compound of Formula (I) is present in an amount of about 0.1 mol % to about 0.25 mol %. In some embodiments, the compound of Formula (I) is present in an amount of about 0.03 mol %. In some embodiments, the compound of Formula (I) is present in an amount of about 0.2 mol %. Without wishing to be bound by any particular theory, it is believed that the increased catalytic activity of the compound of Formula (I) allows for the use of smaller amounts of the catalyst as compared to other palladium-based catalysts that have lower catalytic activity. For example, the amount of the compound of Formula (I) can be less than or about 1 mol %, which is less than the amount of about 1 mol % to about 5 mol % required by other palladium-based catalysts with lower catalytic activity.

In some embodiments of the methods of producing alkynones as described in the present disclosure, any suitable CO source can be used. In some embodiments, the CO source is carbon monoxide gas.

The alkynones of the present disclosure have utility as precursors in the synthesis of products such as anti-fungal agents, as well as products useful for treating neurodegenerative, inflammatory, and infectious diseases, diabetes, and cancer. The alkynones of the present disclosure can also be used in the polymer industry and petrochemical industry. In some embodiments, the method produces an alkynone having the formula:

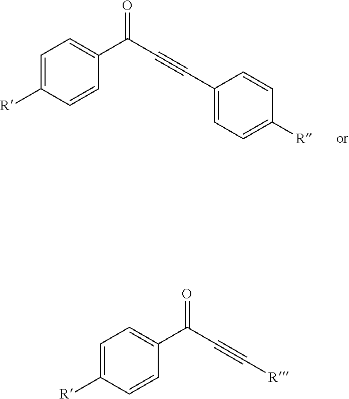

or a pharmaceutically acceptable salt thereof.

In some embodiments, the alkynone is a compound having the formula

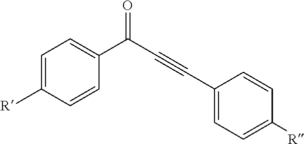

or a pharmaceutically acceptable salt thereof.

In some embodiments, the alkynone is a compound having the formula

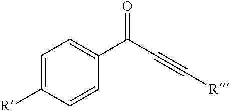

or a pharmaceutically acceptable salt thereof.

Methods of Preparing Chromones

The compounds of Formula (I) of the present disclosure are useful as catalysts. For example, the compounds of Formula (I) can be used as catalysts for the synthesis of chromones, including substituted chromones such as flavones. In some embodiments, the compounds of Formula (I) are used as a catalyst in a cyclocarbonylative Sonogashira coupling reaction. In some embodiments, the cyclocarbonylative Sonogashira coupling reaction is between an aryl halide, such as 2-iodophenol, and an alkyne, such as an aryl alkyne, alkyl alkyne, or dialkyne.

In some embodiments, the chromones of the present disclosure are prepared according to the general scheme presented in Scheme 4, where R^{a} can be any substituent, and R^{b}—C≡C can be an aryl alkyne, alkyl alkyne, or dialkyne.

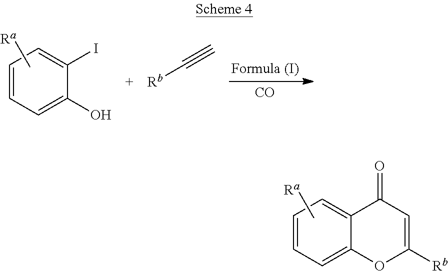

Thus, provided in the present disclosure is a method of preparing a chromone, the method including contacting an optionally substituted 2-iodophenol and an alkyne with a compound of Formula (I) as described in the present disclosure in the presence of a CO source.

In some embodiments, the method includes contacting an optionally substituted 2-iodophenol and an alkyne with a compound of Formula (I) as described in the present disclosure in the presence of a CO source. In some embodiments, the optionally substituted 2-iodophenol is a compound having the formula:

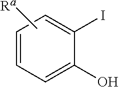

wherein R^{a} is selected from H, C₁-C₆ alkyl, —O—(C₁-C₆ alkyl), —C(═O)C₁-C₆ alkyl, and aryl.

In some embodiments, R^{a} is H.

In some embodiments, the method includes contacting an optionally substituted 2-iodophenol and an alkyne with a compound of Formula (I) as described in the present disclosure in the presence of a CO source. In some embodiments, the alkyne is a compound having the formula:

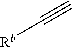

wherein R^{b} is selected from aryl, C₁-C₆ alkyl, and C₃-C₉ cycloalkyl.

In some embodiments, the alkyne is a compound having the formula:

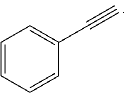

In some embodiments of the method of producing a chromone, the compound of Formula (I) is a compound of Formula (I) of the present disclosure. In some embodiments, the compound of Formula (I) is selected from:

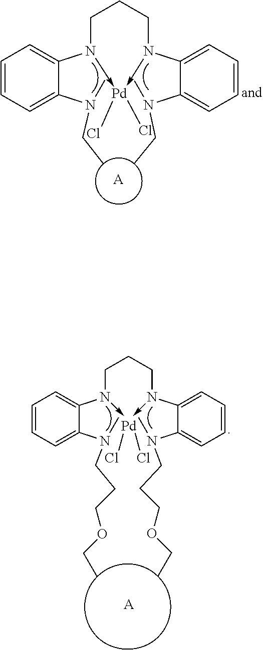

The compounds of Formula (I) have high catalytic efficiency and activity and allow for low catalyst loading. In some embodiments, less than or about 2 mol %, or less than or about 1 mol % of the compound of Formula (I) is required to catalyze a reaction, such as a cyclocarbonylative Sonogashira coupling reaction. In some embodiments, the amount of catalyst compound of Formula (I)) used in the cyclocarbonylative Sonogashira coupling reaction is about 0.01 mol % to about 2 mol %, such as about 0.01 mol % to about 1.5 mol %, about 0.01 mol % to about 1 mol %, about 0.01 mol % to about 0.99 mol %, about 0.01 mol % to about 0.9 mol %, about 0.01 mol % to about 0.8 mol %, about 0.01 mol % to about 0.7 mol %, about 0.01 mol % to about 0.6 mol %, about 0.01 mol % to about 0.5 mol %, about 0.01 mol % to about 0.4 mol %, about 0.01 mol % to about 0.3 mol %, about 0.01 mol % to about 0.2 mol %, about 0.01 mol % to about 0.1 mol %, about 0.01 mol % to about 0.05 mol %, about 0.01 mol % to about 0.03 mol %, about 0.03 mol % to about 2 mol %, about 0.03 mol % to about 1.5 mol %, about 0.03 mol % to about 1 mol %, 0.03 mol % to about 0.99 mol %, about 0.03 mol % to about 0.9 mol %, about 0.03 mol % to about 0.8 mol %, about 0.03 mol % to about 0.7 mol %, about 0.03 mol % to about 0.6 mol %, about 0.03 mol % to about 0.5 mol %, about 0.03 mol % to about 0.4 mol %, about 0.03 mol % to about 0.3 mol %, about 0.03 mol % to about 0.2 mol %, about 0.03 mol % to about 0.1 mol %, about 0.03 mol % to about 0.05 mol %, about 0.05 mol % to about 2 mol %, about 0.05 mol % to about 1.5 mol %, about 0.05 mol % to about 1 mol %, 0.05 mol % to about 0.99 mol %, about 0.05 mol % to about 0.9 mol %, about 0.05 mol % to about 0.8 mol %, about 0.05 mol % to about 0.7 mol %, about 0.05 mol % to about 0.6 mol %, about 0.05 mol % to about 0.5 mol %, about 0.05 mol % to about 0.4 mol %, about 0.05 mol % to about 0.3 mol %, about 0.05 mol % to about 0.2 mol %, about 0.05 mol % to about 0.1 mol %, about 0.1 mol % to about 2 mol %, about 0.1 mol % to about 1.5 mol %, about 0.1 mol % to about 1 mol %, 0.1 mol % to about 0.99 mol %, about 0.1 mol % to about 0.9 mol %, about 0.1 mol % to about 0.8 mol %, about 0.1 mol % to about 0.7 mol %, about 0.1 mol % to about 0.6 mol %, about 0.1 mol % to about 0.5 mol %, about 0.1 mol % to about 0.4 mol %, about 0.1 mol % to about 0.3 mol %, about 0.1 mol % to about 0.2 mol %, about 0.2 mol % to about 2 mol %, about 0.2 mol % to about 1.5 mol %, about 0.2 mol % to about 1 mol %, 0.2 mol % to about 0.99 mol %, about 0.2 mol % to about 0.9 mol %, about 0.2 mol % to about 0.8 mol %, about 0.2 mol % to about 0.7 mol %, about 0.2 mol % to about 0.6 mol %, about 0.2 mol % to about 0.5 mol %, about 0.2 mol % to about 0.4 mol %, about 0.2 mol % to about 0.3 mol %, about 0.3 mol % to about 2 mol %, about 0.3 mol % to about 1.5 mol %, about 0.3 mol % to about 1 mol %, 0.3 mol % to about 0.99 mol %, about 0.3 mol % to about 0.9 mol %, about 0.3 mol % to about 0.8 mol %, about 0.3 mol % to about 0.7 mol %, about 0.3 mol % to about 0.6 mol %, about 0.3 mol % to about 0.5 mol %, about 0.3 mol % to about 0.4 mol %, about 0.4 mol % to about 2 mol %, about 0.4 mol % to about 1.5 mol %, about 0.4 mol % to about 1 mol %, 0.4 mol % to about 0.99 mol %, about 0.4 mol % to about 0.9 mol %, about 0.4 mol % to about 0.8 mol %, about 0.4 mol % to about 0.7 mol %, about 0.4 mol % to about 0.6 mol %, about 0.4 mol % to about 0.5 mol %, about 0.5 mol % to about 2 mol %, about 0.5 mol % to about 1.5 mol %, about 0.5 mol % to about 1 mol %, 0.5 mol % to about 0.99 mol %, about 0.5 mol % to about 0.9 mol %, about 0.5 mol % to about 0.8 mol %, about 0.5 mol % to about 0.7 mol %, about 0.5 mol % to about 0.6 mol %, about 0.6 mol % to about 2 mol %, about 0.6 mol % to about 1.5 mol %, about 0.6 mol % to about 1 mol %, 0.6 mol % to about 0.99 mol %, about 0.6 mol % to about 0.9 mol %, about 0.6 mol % to about 0.8 mol %, about 0.6 mol % to about 0.7 mol %, about 0.7 mol % to about 2 mol %, about 0.7 mol % to about 1.5 mol %, about 0.7 mol % to about 1 mol %, 0.7 mol % to about 0.99 mol %, about 0.7 mol % to about 0.9 mol %, about 0.7 mol % to about 0.8 mol %, about 0.8 mol % to about 2 mol %, about 0.8 mol % to about 1.5 mol %, about 0.8 mol % to about 1 mol %, 0.8 mol % to about 0.99 mol %, about 0.8 mol % to about 0.9 mol %, about 0.9 mol % to about 2 mol %, about 0.9 mol % to about 1.5 mol %, about 0.9 mol % to about 1 mol %, 0.9 mol % to about 0.99 mol %, about 1 mol % to about 2 mol %, about 1 mol % to about 1.5 mol %, about 1.5 mol % to about 2 mol %, or about 0.01 mol %, about 0.03 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.55 mol %, about 0.6 mol %, about 0.65 mol %, about 0.7 mol %, about 0.75 mol %, about 0.8 mol %, about 0.85 mol %, about 0.9 mol %, about 0.95 mol %, about 0.99 mol %, about 1 mol %, about 1.5 mol %, or about 2 mol %. In some embodiments, the compound of Formula (I) is present in an amount of about 0.01 mol % to about 2 mol %. In some embodiments, the compound of Formula (I) is present in an amount of about 0.01 mol % to about 1.5 mol %. In some embodiments, the compound of Formula (I) is present in an amount of about 0.01 mol % to about 1 mol %. In some embodiments, the compound of Formula (I) is present in an amount of about 0.01 mol % to about 0.5 mol %. In some embodiments, the compound of Formula (I) is present in an amount of about 0.01 mol % to about 0.05 mol %. In some embodiments, the compound of Formula (I) is present in an amount of about 0.1 mol % to about 0.25 mol %. In some embodiments, the compound of Formula (I) is present in an amount of about 0.03 mol %. In some embodiments, the compound of Formula (I) is present in an amount of about 0.2 mol %. Without wishing to be bound by any particular theory, it is believed that the increased catalytic activity of the compound of Formula (I) allows for the use of smaller amounts of the catalyst as compared to other palladium-based catalysts that have lower catalytic activity. For example, the amount of the compound of Formula (I) can be less than or about 2 mol %, less than or about 1.5 mol %, or less than or about 1 mol %, which can be less than the amount required by other palladium-based catalysts with lower catalytic activity.

In some embodiments of the methods of producing chromones as described in the present disclosure, any suitable CO source can be used. In some embodiments, the CO source is carbon monoxide gas.

The chromones of the present disclosure have utility as precursors in the synthesis of products such as anti-fungal agents, as well as products useful for treating neurodegenerative, inflammatory, and infectious diseases, diabetes, and cancer. The chromones of the present disclosure can also be used in the polymer industry and petrochemical industry. In some embodiments, the method produces a chromone having the formula:

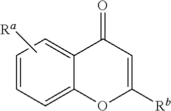

or a pharmaceutically acceptable salt thereof.

In some embodiments, the method produces a chromone having the formula:

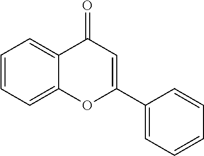 [SMILES: O=c1cc(-c2ccccc2)oc2ccccc12]

or a pharmaceutically acceptable salt thereof.

Recycling the Catalysts

In any of the methods described in the present disclosure, the method can further comprise separating the catalyst composition from the reaction product (for example, an alkynones or chromones) to recover the catalyst composition. In some embodiments, the method further comprises reusing the catalyst composition after recovering the catalyst composition from a reaction product. In some embodiments, the method comprises using the catalyst composition in at least two reaction cycles. In some embodiments, the method comprises using the catalyst composition in at least two reaction cycles with a less than about 5% decrease in at least one selected from the group consisting of catalytic activity, a molar yield of the reaction product (for example, an alkynones or a chromones), or a weight percentage of Pd metal relative to the total weight of the catalyst composition.

The solid-supported catalyst can be removed from the reaction product by any suitable method. In some embodiments, the solid-supported catalyst is separated from the reaction product by removing the bag of solid-supported catalyst, dialysis in a solvent, or using a micro-filter or a paper filter. The phrase “recycling the solid-supported catalyst” refers to a process whereby the solid-supported catalyst or catalyst composition is first washed by an organic solvent, dried, and then added to a new batch of reactants (either for the same or a different type of coupling reaction). Organic solvents suitable for washing the solid-supported catalyst and/or dialysis include, but are not limited to, methanol, acetone, ethanol, tetrahydrofuran, acetonitrile, dichloromethane, ether, glycol ether, acetamide, dimethyl acetamide, dimethyl sulfoxide, and combinations thereof. The solid-supported catalyst or catalyst composition can be dried in vacuum, with or without heating, for example, the catalyst can be dried in a vacuum oven. The dried solid-supported catalyst or catalyst composition can be stored in a desiccator until the next use.

In some embodiments, the catalyst composition has a turnover number of about 1500 to about 2500, such as about 1500 to about 2000, or about 1700 to about 2000, after at least about 8 reaction cycles, at least about 10 reaction cycles, at least about 12 reaction cycles, at least about 15 reaction cycles, at least about 20 reaction cycles, or at least about 30 cycles.

In some embodiments, the catalyst composition has a turnover frequency of about 200 to about 1500 cycles per hour, about 200 to about 1000 cycles per hour, or about 200 to about 500 cycles per hour, after at least about 8 reaction cycles, at least about 10 reaction cycles, at least about 12 reaction cycles, at least about 15 reaction cycles, at least about 20 reaction cycles, or at least about 30 cycles.

In some embodiments, the solid-supported catalyst or catalyst composition is recycled for at least 2 reaction cycles, such as at least about 5 cycles, at least about 8 cycles, at least about 10 cycles, at least about 12 cycles, at least about 15 cycles, at least about 20 cycles, or at least about 30 cycles. In some embodiments, the catalyst composition loses less than about 5 wt %, such as less than about 4 wt %, less than about 3 wt %, less than about 2 wt %, less than about 1 wt %, less than about 0.5 wt %, or less than about 0.1 wt % of palladium (based on an initial amount of palladium present in the solid-supported catalyst and the total weight of the catalyst composition), after the solid-supported catalyst is used for at least 2 reaction cycles. In some embodiments, the catalyst composition loses less than about 5 wt %, such as less than about 4 wt %, less than about 3 wt %, less than about 2 wt %, less than about 1 wt %, less than about 0.5 wt %, or less than about 0.1 wt % of palladium (based on an initial amount of palladium present in the solid-supported catalyst and the total weight of the catalyst composition), after the solid-supported catalyst is used for at least 2 reaction cycles, at least about 5 cycles, at least about 8 cycles, at least about 10 cycles, at least about 12 cycles, at least about 15 cycles, at least about 20 cycles, or at least about 30 cycles.

In some embodiments, the yield of the product from the coupling reaction decreases by less than about 20%, such as less than about 10%, less than about 5%, or less than about 2% after the solid-supported catalyst is used for at least 2 reaction cycles. In some embodiments, the yield of the product from the coupling reaction decreases by less than about 20%, such as less than about 10%, less than about 5%, or less than about 2% after the solid-supported catalyst is used for at least 2 reaction cycles, at least about 5 cycles, at least about 8 cycles, at least about 10 cycles, at least about 12 cycles, at least about 15 cycles, at least about 20 cycles, or at least about 30 cycles.

In some embodiments, the turnover number and the turnover frequency of the solid-supported catalyst or catalyst composition decrease by less than about 10%, such as less than about 5%, or less than about 2% after the solid-supported catalyst is used for at least 2 reaction cycles.

In some embodiments, the turnover number and the turnover frequency of the solid-supported catalyst or catalyst composition decrease by less than about 10%, such as less than about 5%, or less than about 2% after the solid-supported catalyst is used for at least 2 reaction cycles, at least about 5 cycles, at least about 8 cycles, at least about 10 cycles, at least about 12 cycles, at least about 15 cycles, at least about 20 cycles, or at least about 30 cycles

### EXAMPLES

### Example 1— Synthesis of Solid-Supported Palladium(II)—N-Heterocyclic Carbene Catalysts

Palladium(II)—N-heterocyclic carbene catalysts Pd-NHC₁ and Pd-NHC₂ were prepared in several steps from 1H-benzo[d]imidazole.

Synthesis of Bridged Catalyst Ligands

Bridged catalyst ligands NHC₁ and NHC₂ were prepared according to Schemes 5 and 6, respectively.

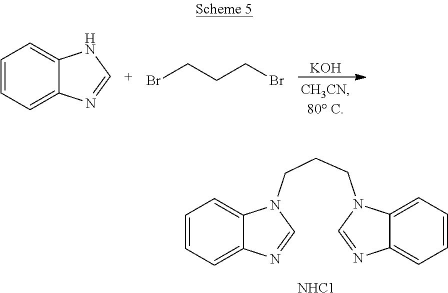

1,3-bis(1H-benzo[d]imidazol-1-yl)propane (NHC1). 1,3-bis(1H-benzo[d]imidazol-1-yl)propane (NHC₁) was prepared from the N-alkylation of 2 mol equivalent of benzimidazole with 1 mol equivalent of 1,3-dibromopropane in the presence of potassium hydroxide (Scheme 5).

Briefly, a dry and clean round bottom flask was charged with benzimidazole (10.0 mmol), an excess amount of 1,3-dibromopropane (5.50 mmol), and potassium hydroxide (20.0 mmol) as a base. Then, 100 mL of distilled acetonitrile was added and the mixture was stirred at 80° C. for 24 hrs. The reaction was monitored by TLC (50% hexane:50% ethyl acetate) until all benzimidazole was consumed. After the reaction was complete, the solvent was evaporated under vacuum using a rotary evaporator. Oily products were obtained as residues, which were purified by extracting three times with 30 mL of ethyl acetate and 20 mL of distilled water. The separated organic layers were washed several times by n-hexane, the aqueous layer was separated then washed with ethyl acetate. NHC₁ was isolated as a sticky brown oil product (isolated yield=73%).

¹H NMR (500 MHz, DMSO-d6) δ (ppm): 8.14 (s, 2H, NCHN), 7.65 (2H, m, Ar—H), 7.63 (2H, m, Ar—H), 7.53-7.35 (4H, m, Ar—H), 4.62 (4H, t, ³J=7.32 Hz, NCH) 2.02 (2H, m, NCH₂(CH₂)₂); ¹³C NMR (125 MHz, DMSO-d6) δ (ppm): 143.1 (NCN), 133.8, 122.7, 121.7, 119.7, 110.5, (Ar—H), 41.9 (NCH₂), 29.8 [NC(CH₂)₂]; Anal. Calcd for C₁₇H₁₆N₄ (276.34), C, 73.89; H, 5.84, N; 20.27; Found: C, 74.83; H, 5.24; N, 19.93.

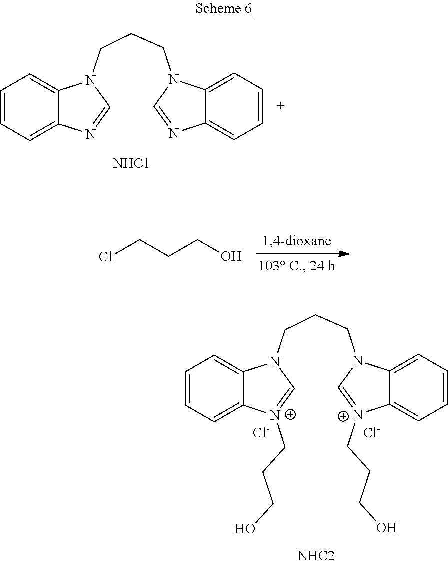

1,1′-(propane-1,3-diyl)bis(3-(3-hydroxypropyl)-1H-benzo[d ]imidazol-3-ium) chloride (NHC2). 1,1′-(Propane-1,3-diyl)-bis(3-(2-hydroxyethyl)-1H-benzo[d]imidazol-3-ium) chloride salt ligand precursor NHC₂ was synthesized by the direct alkylation of 1,3-bis(1H-benzo[d]imidazol-1-yl) propane (NHC₁) with 3-chloropropanol. The formation of the hydroxyl propyl functionalized bridged bis(benzimidazolium chloride) salt was confirmed by the presence of a downfield singlet peak at δ10.05 ppm, in its proton NMR spectrum, which is assigned to C-2 protons of the benzimidazole rings.

Briefly, 1,3-bis(1H-benzo[d]imidazol-1-yl) propane (NHC₁) (2.5 mmol) and 3-chloropropanol (5.0 mmol) were added into a dried 100-mL round bottom flask. The mixture was refluxed in 25 mL of 1,4-dioxane solvent with stirring at 103° C. for 24 hrs. The product appeared as a pink-white salt precipitate. After decanting the solvent, the product was washed three times using 10 mL of 1,4-dioxane followed by 10 mL of toluene to remove any traces of the starting materials. The product was dried under vacuum and collected as a white precipitate (isolated yield=87%; pink-white product). The characterization of the 1,1′-(propane-1,3-diyl)bis(3-(2-hydroxyethyl)-1H-benzo[d]imidazol-3-ium) chloride salt was conducted using various spectroscopic techniques including ¹H NMR, ¹³C NMR, and elemental analysis.

¹H NMR (500 MHz, DMSO-d6) δ (ppm): 9.92 (s, 2H, NCHN), 8.13-8.06 (4H, m, Ar—
H), 7.79-7.67 (4H, m, Ar—H), 4.71-4.68 (4H, m, NCH₂) 4.63-4.53 (4H, m, NCH₂), 3.4 (4H, m 2CH₂OH overlapping with H₂O signal), 2.76 (2H, broad OH), 2.6 (2H, m, CH₂), 2.06-2.04 (4H, m, 2x CH₂); ¹³C NMR (125 MHz, DMSO-d6) δ (ppm): 142.51 (NCN), 131.3, 131.2, 131.1, 126.8, 126.6, 113.8, 113.7, (Ar—H), 66.9 (NCH₂), 57.6 (NCH₂), 44.5, 44.1 (NCH₂)₂, 31.3, 30.8, 28.7, 28.3CCH₂; Anal. Calcd for C₂₃H₃₀N₄O₂Cl₂ (465.41): C, 59.36; H, 6.50; N, 12.04; Found: C, 58.63; H, 5.54; N, 11.73.

Synthesis of Solid-Supported Bridged Catalyst Ligands and Solid-Supported Bridged Palladium(II) Catalysts

Solid-supported bridged catalyst ligands S—NHC₁ and S—NHC₂ were prepared from NHC₁ and NHC₂, respectively, then further reacted with palladium(II) acetate to form palladium(II) catalysts Pd-NHC₁ and Pd-NHC₂ as shown in Schemes 7 and 8, respectively. “PS” represents “polystyrene” in Schemes 7 and 8.

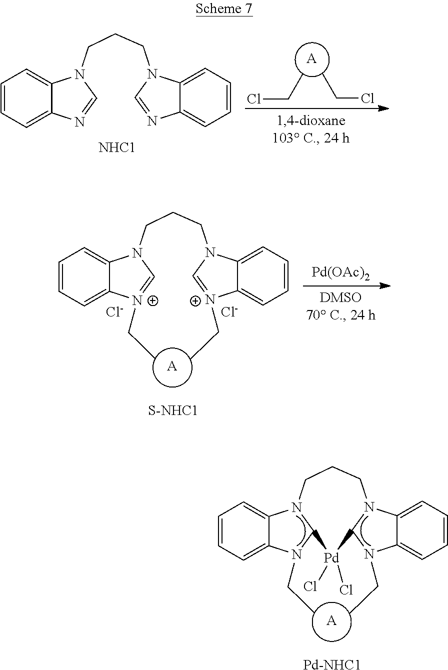

Solid-supported palladium(II)—NHC catalyst (Pd-NHC1). The propylene-bridged NHC ligand 1, l′-(propane-1,3-diyl)-bis(3-(3 (benzyl)-1H-benzo[d]imidazol-3-ium) chloride (NHC₁) was reacted with benzyl chloride-functionalized Merrifield's resin support to obtain the immobilized propylene bridged-bis(NHC) ligand S—NHC₁ via N—C alkylation. S—NHC₁ was reacted further with palladium(II) acetate to afford the supported palladium(II)—NHC catalyst Pd-NHC₁.

1,3-Bis(1H-benzo[d]imidazol-1-yl)ium chloride propane supported on Merrifield's resin (S—NHC-1)

Merrifield's resin (0.12 g) and 1.5 mmol (0.414 g) of 1,3-bis(1H-benzo[d]imidazol-1-yl) propane (NHC₁) were introduced into a cleaned and dried 10 mL round bottom flask. The mixture was refluxed in 25 mL of dimethyl sulfoxide (DMSO) under stirring at 120° C. for 48 hrs. After completion of the reaction, the dark brown precipitate was collected by filtration and washed three times successively with 10 mL of DMSO, distilled water, and dichloromethane (DCM) to remove any traces of the starting materials. The golden-brown solid (S—NHC-1) was dried under vacuum and characterized using different spectroscopic techniques including CP-MAS NMR, FT-IR, TGA, and SEM (isolated yield=83%).

¹³C NMR: δ (ppm): 40.5, 127.9, 134.8, 145.9, 180.7 (Pd-C) carbene signal; IR: vₘₐₓ (cm⁻¹) 3430 (broad), 3024, 2921, 1960, 1938, 1602, 1565, 1490, 1448, 1022, 952, 700, 548; Metal loading from ICP-MS: 1.2% corresponding to 0.12 mmol/g.

Merrifield's Resin-Supported Dichlorido Bis(1,3-Bis(1H-Benzo[d]imidazol-1-Yl) Propane Palladium(II) Catalyst (Pd-NHC₁)

The Merrifield resin supported-propylene bridged bis(N-heterocyclic carbene) ligand precursor (S—NHC-1) (3.0 mmol, 1.0 g) was stirred in DMSO for 30 minutes. Palladium(II) acetate (3.0 mmol, 0.67 g) was then added and the resulting mixture was stirred at 70° C. for 24 hrs. The dark solid product was collected by filtration and washed thoroughly with THF, toluene, 1,4-dioxane, DCM, ethyl acetate, diethyl ether, and H₂O followed by a drying step under vacuum. Isolated yield=83%.

¹³C NMR: δ (ppm): 40.5, 127.9, 134.8, 145.9, 180.7 (Pd-C) carbene signal; IR: vₘₐₓ (cm⁻¹) 3430 (broad), 3024, 2921, 1960, 1938, 1602, 1565, 1490, 1448, 1022, 952, 700, 548; Metal loading from ICP-MS: 1.2% corresponding to 0.12 mmol/g.

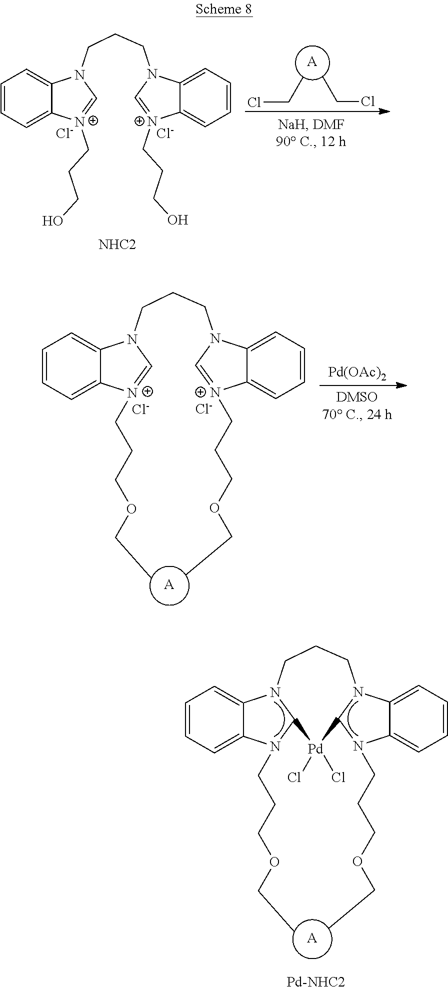

Solid-supported palladium(II)—NHC catalyst (Pd-NHC2). The hydroxyl-functionalized propylene-bridged NHC ligand NHC₂ was reacted with benzyl chloride-functionalized Merrifield's resin support to afford the immobilized ligand S—NHC₂ via an ether linkage between the ligand and the support. S—NHC₂ was further reacted with palladium(II) acetate to yield the supported palladium(II)—NHC catalyst Pd-NHC₂. The ether bond is considered suitable for linking the NHC ligand to the support as the ether link is particularly stable under the reaction conditions, which is expected to prevent the leaching of the ligand during the catalytic application.

By contrast, many other functional groups, such as esters and amides, can be easily hydrolyzed under the standard conditions of coupling reactions that encounter the use of relatively strong bases such as KOH and K₂CO₃.

Supported 1,1′-(Propane-1,3-Diyl)Bis(3-(3-(Benzyloxy)Propyl)-1H-Benzo[d]Imidazol-3-Ium) Chloride on Merrifield's Resin (S—NHC₂)

In a dry flask, NaH (0.50 mmol) was added in one portion to a stirred solution of 1,1′-(propane-1,3-diyl)bis(3-(2-hydroxypropyl)-1H-benzo[d]imidazol-3-ium) chloride (1.5 mmol) in dry DMF. The mixture was stirred for 2 hrs at room temperature under nitrogen atmosphere. Merrifield's resin (1.2 g) was added and the mixture was stirred at 90° C. for 12 hrs. The solid product was filtered and washed successively with DMSO, DMF, methanol, water, acetone, and dichloromethane. The product was dried at room temperature under vacuum and the supported propylene bridged bis(NHC) on Merrifield's resin (S—NHC₂) was obtained as a pre-ligand. Physical and spectroscopic techniques were used to characterize the (S—NHC₂) supported pre-ligand salt such as NMR, FT-IR, SEM, TGA, and ICP-MS. Isolated yield=93% (golden-brown solid).

CP-MAS ¹³C NMR: δ28.2, 41.1, 44.8, 114.1, 128.1, 130.6, 145.3; IR: vₘₐₓ (cm⁻¹) 3431 (broad), 3024, 2922, 1639, 1565, 1491, 1448, 1206, 1022, 952, 756, 699.

Merrifield's Resin-Supported Dichlorido Bis(1,1′-(Propane-1,3-Diyl)Bis(3-(3-(Benzyloxy) Propyl)-1H-Benzo[d]imidazol-1-Yl) Palladium(II) Catalyst (Pd-NHC₂)

The Merrifield resin-supported-propylene bridged bis(N-heterocyclic carbene) ligand precursor (S—NHC-2) (3.0 mmol, 1.0 g) was stirred in DMSO for 30 minutes. Palladium(II) acetate (3.0 mmol, 0.67 g) was then added and the resulting mixture was stirred at 70° C. for 24 hrs. The dark solid product was collected by filtration and washed thoroughly with THF, toluene, 1,4-dioxane, DCM, ethyl acetate, diethyl ether, and H₂O followed by a drying step under vacuum. Isolated yield=96%.

CP-MAS ¹³C NMR δ (ppm): 24.5, 40.8, 46.1, 112.3, 127.6, 134.0, 146.5, 178.4 (Pd-C) carbene signal; IR: vₘₐₓ(cm⁻¹) 3438, 3022, 2918, 2850, 1696, 1601, 1491, 1449, 1307, 1327, 1211, 1176, 1022, 829, 754, 699, 544; Metal loading from ICP-MS: 8.7% corresponding to 0.83 mmol/g.

### Example 2—Carbonylative Sonogashira Coupling Reactions

The solid-supported palladium(II)—N-heterocyclic carbene catalysts Pd-NHC₁ and Pd-NHC₂, prepared according to Example 1, were used in carbonylative Sonogashira coupling reactions to produce alkynones. The Pd-NHC catalysts displayed high catalytic activity with low catalyst loading and exhibited a high degree of recyclability. The reactions required only 0.5 mol % of the Pd-NHC catalyst and produced alkynones in high yield.

A series of aryl alkynones was synthesized by reacting an aryl iodide (1a-1f) with an alkyne (2a-2c or 4a-4b) in the presence of 0.5 mol % Pd-NHC₂ catalyst, triethylamine (Et₃N; 2.0 eqv.), and CO (200 psi) in toluene (3 mL) at 100° C. for 12 hours, as shown in Schemes 9a and 9b and Table 1. Alkynones 3aa-3fa and 5ba-5bb were produced in excellent yields (79-99%) via the carbonylative Sonogashira coupling reaction.

The general procedure is as follows. The carbonylative Sonogashira coupling reactions were conducted in a 45 mL stainless steel autoclave equipped with a glass liner, a gas inlet valve and a pressure gauge. The immobilized bridged-bis(N-heterocyclic carbene) palladium(II) catalyst (0.0050 mmol) was placed in a dialysis bag, and aryl iodide (1.0 mmol), alkyne (1.2 mmol), Et₃N as a base (2.0 mmol), and a solvent (3 mL) were introduced into the glass liner. The latter was then carefully placed in the autoclave. The autoclave was vented three times with carbon monoxide then pressurized to 200 psi of CO. The mixture was heated to the required temperature and stirred for the required time. After completion of the reaction, the mixture was cooled down to room temperature and the CO excess was vented slowly and carefully under a fume hood. The dialysis bag with catalyst was removed and dialyzed in acetonitrile to extract the products. The solid catalyst in the dialysis bag was dried in the oven at 100° C. to be reused in the next experiments. The organic mixture was extracted three times with 5 mL of distilled water and 10 mL of ethyl acetate. The ethyl acetate extracts were combined and concentrated in a rotary evaporator under reduced pressure. Several techniques were used to fully characterize the final carbonyl products including ¹H, ¹³C NMR, GC and GC-MS.

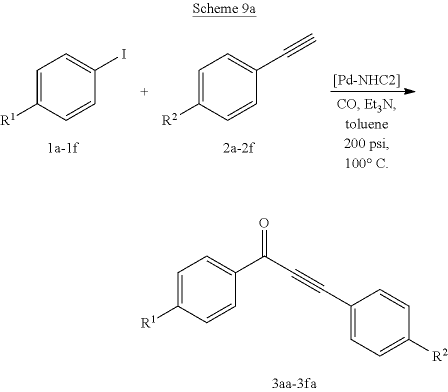

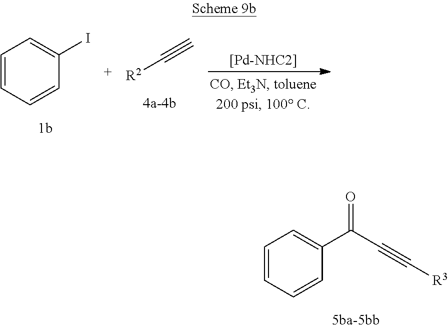

TABLE 1

| | Aryl | Aryl | Carbonylative | Isolated |
|---|---|---|---|---|
| | Iodide | Alkyne | Sonogashira | Yield |
| Entry | 1 | 2 | 3 | (%) |
| 1 | 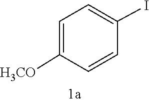 | 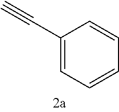 | 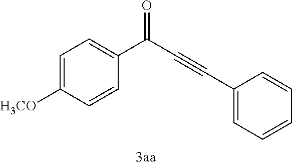 [SMILES: COc1ccc(C(=O)C#Cc2ccccc2)cc1] | 99 |
| 2 | 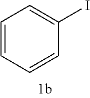 | 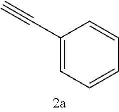 | 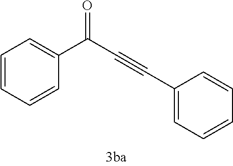 [SMILES: O=C(C#Cc1ccccc1)c1ccccc1] | 93 |
| 3 | 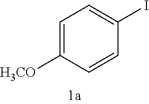 | 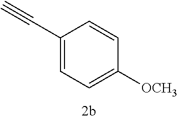 | 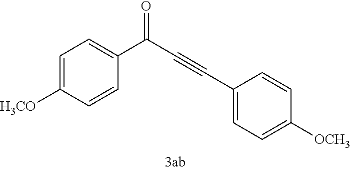 [SMILES: COc1ccc(C#CC(=O)c2ccc(OC)cc2)cc1] | 92 |
| 4 | 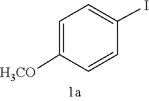 | 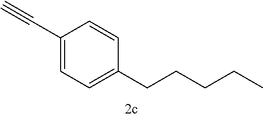 | 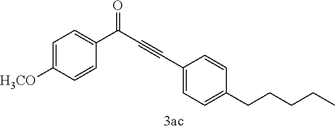 [SMILES: CCCCCc1ccc(C#CC(=O)c2ccc(OC)cc2)cc1] | 93 |
| 5 | 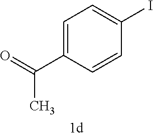 | 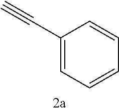 | 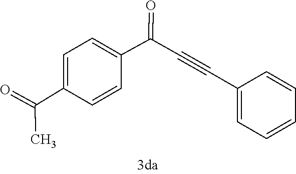 [SMILES: CC(=O)c1ccc(C(=O)C#Cc2ccccc2)cc1] | 90 |
| 6 | 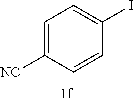 | 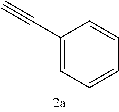 | 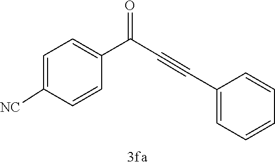 [SMILES: N#Cc1ccc(C(=O)C#Cc2ccccc2)cc1] | 92 |
| 7 | 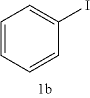 | 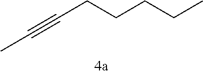 | 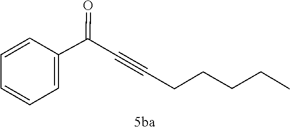 | 79 |
| 8 | 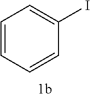 | 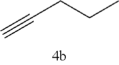 | 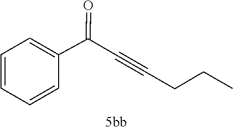 | 83 |

### Example 3—Recycling Ability of the Solid-Supported Palladium(II)—N-Heterocyclic Carbene Catalysts

Carbonylative Sonogashira Reactions-Catalyst Recycling

The recycling ability of the supported catalyst Pd-NHC₂ was evaluated in a carbonylative Sonogashira reaction such as described in Example 2. The supported catalyst Pd-NHC₂ could be recycled up to twelve times without bearing a significant loss in the catalytic activity. The turnover number of Pd-NHC₂ was estimated for the 12 cycles as 2297, while the turnover frequency was estimated as 192 cycles/h. In order to confirm the activity and recycling ability of the supported catalyst, a carbonylative Sonogashira coupling reaction between 4-iodoanisole and phenylacetylene was conducted, where the ratio of 4-iodoanisole (12.0 mmol) to supported Pd-NHC₂ catalyst (0.005 mmol) was equal to 2364 for 12 hours. A complete conversion of 4-iodoanisole and excellent isolated yield (99%) was obtained for the Pd-NHC₂ catalyst. The results are shown in Table 2.

TABLE 2

| | Isolated |
|---|---|
| Cycle | yield (%) |
| 1 | 99 |
| 2 | 99 |
| 3 | 98 |
| 4 | 99 |
| 5 | 97 |
| 6 | 94 |
| 7 | 92 |
| 8 | 94 |
| 9 | 94 |
| 10 | 94 |
| 11 | 94 |
| 12 | 92 |

Cyclocarbonylative Sonogashira Reactions

The recycling ability of the supported catalyst Pd-NHC₂ was evaluated in a cyclocarbonylative Sonogashira reaction. A cyclocarbonylative Sonogashira reaction between 2-iodophenol with phenylacetylene with a Pd-NHC₂ catalyst loading of 1.5 mol % was performed as shown in Scheme 10.

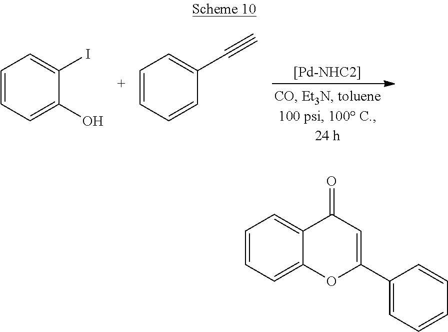

The general procedure is as follows and can be used for coupling reactions between 2-iodophenols and aryl terminal alkynes. Chromones were synthesized via cyclocarbonylative Sonogashira coupling reactions that were accomplished in a 45 mL stainless steel autoclave equipped with a glass liner, gas inlet valve and pressure gauge. The immobilized bridged-bis(N-heterocyclic carbene) palladium(II) catalyst contained in a dialysis bag (0.015 mmol), substituted 2-iodophenol (0.50 mmol), alkyne (0.55 mmol), base (1.0 mmol) and an anhydrous solvent (2 mL) were charged into the glass liner of the autoclave. Then the autoclave was vented carefully three times with CO then pressurized to 100 psi of CO. The mixture was stirred and heated to the required temperature for a specific period. At the end of the reaction period, the autoclave was cooled down to room temperature and the excess of CO was discharged carefully under a fume hood. The dialysis bag with catalyst was removed and dialyzed in acetonitrile to extract all the products. The solid catalyst in the dialysis bag was dried in the oven at 100° C. to be used in the next experiment. The organic mixture was extracted three times with 5 mL of distilled water and 10 mL of ethyl acetate. The ethyl acetate extracts were combined and concentrated in a rotary evaporator under reduced pressure. Flash chromatography was used to purify the reaction mixture using silica gel and an eluent (pentane/ethyl acetate=7/1) to afford the corresponding chromones or flavones. Various physical and spectroscopic techniques such as ¹H and ¹³C NMR, GC and GC-MS were used to fully characterize the products.

The supported palladium catalyst Pd-NHC₂ could be recycled up to 10 times with only a small loss in the catalytic activity. The turnover number of Pd-NHC₂ was estimated for 10 cycles as 627, while the turnover frequency was estimated as 26/h. In order to confirm the efficiency of Pd-NHC₂, an experiment was conducted using a ratio of 2-iodophenol (10.0 mmol) to Pd-NHC₂ (0.015 mmol) equal to 647 for 24 hours, while the turnover frequencies were found to be 30/h. A complete conversion of 2-iodophenol and excellent isolated yield of product (97%) using Pd-NHC₂ was observed. The results are shown in Table 3.

TABLE 3

| | Isolated |
|---|---|
| Cycle | yield (%) |
| 1 | 97 |
| 2 | 98 |
| 3 | 96 |
| 4 | 96 |
| 5 | 95 |
| 6 | 94 |
| 7 | 92 |
| 8 | 93 |
| 9 | 90 |
| 10 | 89 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (I) 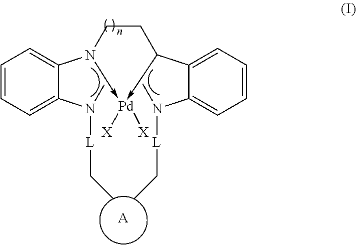
wherein:
L is absent;
X is selected from the group consisting of Cl, Br, and I;
A is a solid support; and
n is 0 to 4.

2.-5. (canceled)

6. The compound of claim 1, wherein Xis Cl.

7. The compound of claim 1, wherein A is Merrifield resin.

8. The compound of claim 1, wherein n is 1 or 2.

9. The compound of claim 1, wherein n is 2.

10. The compound of claim 1, wherein the compound of Formula (I) is:

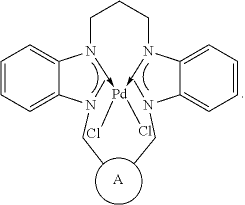

11. The compound of claim 1, which has a turnover number in a range of about 1500 to about 2500 and a turnover frequency of about 200 to about 1500 per hour.

12.-40. (canceled)

41. A compound of Formula (I) 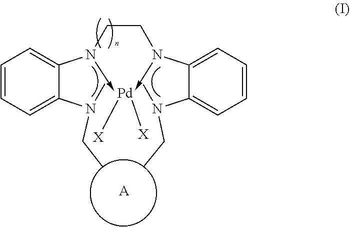
wherein:
L is absent or is selected from the group consisting of —C₁-C₆ alkylene- and —C₁-C₆ alkylene—O—;
X is selected from the group consisting of Cl, Br, and I;
A is Merrifield resin; and
n is 0 to 4.

42. The compound of claim 41, wherein L is —C₁-C₆ alkylene—O—.

43. The compound of claim 42, wherein L is selected from —CH₂—O—, —(CH₂)₂—O—, —(CH₂)₃—O—, —(CH₂)₄—O—, —(CH₂)₅—O—, and —(CH₂)₆—O—.

44. The compound of claim 43, wherein L is —(CH₂)₃—O—.

45. The compound of claim 41, wherein L is absent.

46. The compound of claim 41, wherein X is Cl.

47. The compound of claim 41, wherein n is 1 or 2.

48. The compound of claim 41, wherein n is 2.

49. The compound of claim 41, wherein the compound of Formula (I) is selected from:

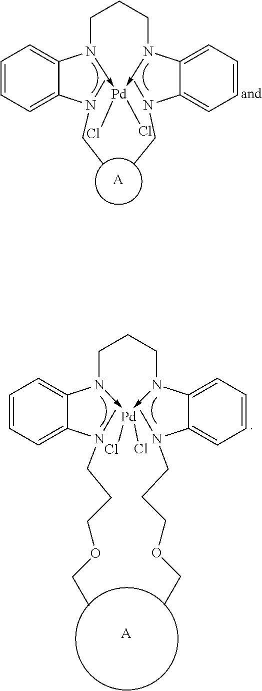

50. The compound of claim 41, which has a turnover number in a range of about 1500 to about 2500 and a turnover frequency of about 200 to about 1500 per hour.
